(19) United States
(12) Patent Application Publication
(10) Pub. No.: US 2012/0309008 A1
(43) Pub. Date: Dec. 6, 2012

(54) Method and Laboratory Apparatus for Processing Laboratory Samples

(75) Inventors: Wolfgang GOEMANN, Hamburg (DE); Kirsten Schicke, Kaltenkirchen (DE)

(73) Assignee: Eppendorf AG, Hamburg (DE)

(21) Appl. No.: 13/463,740

(22) Filed: May 3, 2012

Related U.S. Application Data
(60) Provisional application No. 61/482,961, filed on May 5, 2011.

(30) Foreign Application Priority Data
May 5, 2011 (EP) 11003690.2

(51) Int. Cl. C12Q 1/68; C12M 1/40

(52) U.S. Cl. 435/6.12; 435/289.1

(57) ABSTRACT

The present invention relates to methods for solidifying a sample and to an apparatus, in particular for use in a laboratory, for performing a work step on a sample, comprising a first temperature section adjustable to a first temperature and to which the sample is thermally connectable, a second temperature section with a second temperature, and a solidification device for the solidification of the sample, wherein the sample is transferable at least partially to a solid state of matter such that leakage of the sample is prevented, and wherein the solidification device comprises a trigger device to trigger the solidification of the one sample at a start time, a heat transport device for transporting heat between said first temperature section and said second temperature section during a tempering period starting at said start time, and a control device for controlling the tempering period and the first temperature.

The present invention relates to a method for the processing of laboratory samples and to a laboratory apparatus for the processing of laboratory samples.

A common problem of laboratory apparatus, which perform a work step on a sample, in particular on a plurality of non-solid samples, e.g. liquid samples, is to control the sample quality, e.g. to ensure the purity, molarity or volume of the sample or to prevent cross contamination between samples. Degradation of the sample quality may occur when the sample vessels are mechanically treated, e.g. manually or automatically contacted and moved, by leaking and spilling of liquid drops, or when they are tempered such that condensation effects occur, which e.g. can influence the concentrations of reagents.

To avoid contamination, the sample vessels are usually covered by caps, sealing films or mats, as described for example by EP 1 126 917 B1. After performing a work step on the samples, which can be a physical measurement or treatment, a chemical treatment, an amplification process or the like, and before the removal of said covers, the samples are often centrifuged in order to remove droplets from the covers, because the droplets on wetted covers may lead to cross contamination under the vibrations of the mechanical removal of the covers. Centrifugation, however, complicates the processing of the samples and can state an additional source of error.

It is the object of the present invention to provide a method for improving the reliability of processing at least one sample, in particular the reliability against leakage and the cross-contamination of the sample by spilling of droplets. It is further the object of the present invention to provide an improved apparatus for performing a work step on at least one sample, which provides in particular an improved reliability of processing at least one sample.

The object is met by the method according to claim 1 and the laboratory apparatus according to claim 11. Preferred embodiments are respectively subject matter of the sub-claims.

The laboratory of the present invention can be used in a scientific research laboratory, e.g. chemical, a biological, biochemical, or clinical laboratory or can be used in a non-research laboratory, e.g. an industrial or manufacturing laboratory. Preferably, the laboratory apparatus is a stand-alone device or is preferably part of another apparatus or part of an automated laboratory environment. The apparatus is preferably adapted to manually or automatically perform a work step on at least one sample, in particular on a plurality of samples. The samples are preferably at least partially non-solid, e.g. liquid, gaseous or viscose. Preferably, the samples substantially consist of an aqueous solution of ingredients, preferably of biochemical substances, e.g. substances, which are used in order to perform a polymerase chain reaction (PCR). However, the invention is applicable as well on other non-solids, e.g. solvents, liquids or, generally, pourable matters.

The apparatus (hereinafter, the term “apparatus” is used as a synonym for “laboratory apparatus”) of the present invention is adapted to perform a work step on said at least one sample. Said work step can be referred to as first functionality of the apparatus. Further, said work step can be, for example, the step of thermocycling the samples. Thermocycling is e.g. used in order to perform a PCR on samples. In this case, the apparatus provides preferably the basic functions of a common thermocycler or is basically a thermocycler, or an instrument for PCR, quantitative PCR or real-time quantitative PCR.

Alternatively, the apparatus comprises the substantial functions of a thermostat or is a thermostat and said work step is the process of setting and holding said at least one sample at a target temperature. However, the apparatus according to the present invention is not limited to said type of apparatus but also includes apparatus which perform other work steps as the first functionality, e.g. the work step of shaking the samples in order to mix their reagents, or the work step of centrifuging the samples, or the work step of just holding at least one sample for later use or processing in said laboratory. It is also possible and preferred that said work step is just the solidification of at least one sample.

The apparatus further comprises a first temperature section, which is preferably adjustable to have a first temperature and to which said at least one sample is thermally connectable. The term “thermally connectable” means in this context, that a change of the first temperature influences the temperature of the sample. It is not required but possible and preferred that the sample reaches exactly the first temperature. The “application of the first temperature to the first temperature section” means, either, that the first temperature can be precisely controlled, using electrical circuitry with closed loops, or means that the first temperature is adjusted without the use of closed loops. The apparatus further comprises a second temperature section, which can take a second temperature, wherein preferably a heat flow between the first temperature section and the second temperature section is possible. The second temperature section is preferably located in a heat sink of the apparatus, which can be a metal block with good heat conductivity, which can be equipped with passive heat exchange elements like heating fins and heat pipes or active heat exchange elements like ventilators and which is preferably thermally connected with the environment of the apparatus. It is possible in special cases, but usually not required, that the first and second temperature section, respectively, provide a substantially completely homogeneous distribution of temperature within their respective volume.

The apparatus comprises a solidification device for the solidification of said at least one sample, which is non-solid. Solidification means in the context of this invention that said at least one sample is transferred at least partially to a solid state of matter or that the viscosity of at least a part of the (or the complete) sample is increased. The advantage of said solidification is that the mobility of the sample is reduced such that the probability of leakage or spilling of the sample is reduced. The solidification can utilize different technical effects. Preferably, the sample is exposed to a reduced temperature, e.g. by cooling the sample, such that the sample is transferred at least partially to a solid state of matter or that the viscosity of at least a part of the (or the complete) sample is increased. However, it is also possible to change other parameters for performing the solidification. Further, it is preferred to add a substance or a mixture of substances to the non-solid sample, which achieve the solidification, alone or in combination with the change of physical parameters, e.g. the sample temperature. As an example, agarose or other polymers or gelling agents can be used to transfer an aqueous sample liquid to a gel, or, more generally, a solidification agent, which is combined, e.g. mixed, with the sample, can be used to solidify a non-solid sample, thereby achieving solidification of the non-solid sample. For example, the apparatus may comprise a distribution device for distributing one or more portions of such a substance to a sample in a sample vessel or to several or a plurality of samples.

Therefore, solidification of a non-solid sample can mean, for example, that a liquid sample is transferred to a gel-like, viscous or solid, e.g. crystalline or amorphous state of matter. In another example, a viscous sample, e.g. a liquid or a gel-like sample is solidified by further increasing the viscosity of the sample, e.g. by increasing the viscosity of the sample by, respectively preferably, a factor of 2, 5, 10, 100, 1000, 1000 or a factor between said values or larger factors. Preferably, an aqueous sample is solidified by freezing the sample completely or at least partially, e.g. freezing the aqueous surface of the sample such that a “cap layer” of ice forms on top which prevents leakage. The advantage is not only that solidification prevents leakage and contamination of the sample but that it also slows down chemical reactions in the sample and thus, prevents aging of the sample and conserves the same. The solidification, preferably, is the second functionality of the apparatus according to the present invention.

The configuration of the apparatus according to the present invention with a solidification device preferably is realized in two basic alternate ways. First, an apparatus, which is adapted for a first functionality, i.e. to perform a work step on said at least one sample, can contain said solidification device, which thus shares functional devices with the apparatus. Such shared functional device can be a joint case for holding the components of the apparatus and of the solidification device. Moreover, the apparatus and the solidification device may share at least one device, which performs a task like heat transport or control of functional devices. This leads to a combined apparatus and solidification device with integrated setup. Such a setup is in particular advantageous, if the first functionality of the apparatus requires already a heat transport device, like in the case of the embodiment of the apparatus as thermocycler or thermostat. This way, the integration of components and functions reduces the cost of manufacture and of the operation of the devices.

Second, and in particular preferred according to the present invention, the solidification device can be a modular device, which may be added to the apparatus for performing a work step according to the present invention. The advantage of such a configuration is that even existing apparatus may be equipped with the beneficial functionality of the solidification device according to the present invention. Such a retrofitting of apparatus is an economic solution in particular for expensive apparatus, e.g. apparatus in automated laboratory systems. Preferably, the solidification device is coupled to sub-devices of the apparatus, which are exchangeable. Further preferred, the solidification device is attachable or attached to a lid which covers said at least one sample and which is preferably part of the apparatus. Moreover, the installation of the solidification device is preferably easy and can be performed by un-experienced and/or experienced laboratory users. Further, the apparatus and the solidification device are preferably adapted to be removable connected. In particular, the solidification device can be connected to a sample vessel holder device, in particular to the bottom surface, the top surface or the side faces of a sample block device, e.g. the thermoblock of a thermocycler.

Such a sample vessel holder device may follow the standard specifications, e.g. size, shape, performance or capacity, for a removable sample vessel holder device, e.g. the thermoblock of a commercial thermocycler. Generally, a thermoblock can comprise a plurality of wells, e.g. 24, 96, 384, 1536 or another number of wells, which are generally arranged in an array. The wells can be adapted as holes in the top surface of a sample vessel holder device or sample block device or can be realized by vessel sections of the sample vessel holder device or sample block device, which are arranged side-by-side in an array, and which can be fixed to a bottom plate, which preferably has a flat bottom side. Other suitable configurations of a sample vessel holder device or sample block device or thermoblock are possible. A thermoblock generally serves to transport heat into (or out from) the sample vessels and into the samples, and further preferably serves to store heat for providing a homogeneous temperature incubation of samples. The sample vessel holder device preferably is a thermoblock, which is preferably made of (or at least comprises) a material with high thermal conductivity, like silver, aluminium, copper or the like. Preferably, the wells in the sample vessel holder device and/or the thermoblock are adapted to provide an optimal, e.g. tight, thermal contact to the sample vessels.

The at least one sample vessel is preferably a commercial standard sample vessels, which is specified as standard sample vessels for the respective type of apparatus. The vessels are preferably formed from plastic and can be presented as individual sample vessels or as an array of sample vessels, e.g. microtiter plates with 96, 384, 1536 or a different number of vessels, as PCR plates or other storage plates, preferably according to standard or commercial specifications. It is preferred that means for covering the openings of the sample vessel(s) are provided, which prevent the evaporation of a non-solid sample from the vessel and which prevent contamination of the individual sample vessels by cross-contamination or by other contaminants in the surrounding atmosphere. Such a means for covering can be a sealing foil, which is laminated, glued or otherwise attached to the top side of a microtiter plate. It is further possible that the means for covering comprise a plate for covering one, several or all openings of the sample vessel(s) or comprise strips or mats of individual caps, which are arranged side-by-side and connected to each other.

Preferably, the solidification device according to the present invention is used as stand-alone device, e.g. by providing it with an individual power supply or other independent functional devices. However, it is also possible and preferred that the solidification device, in particular in its modular embodiment, is firmly attached to the apparatus.

Preferably, the apparatus provides sample vessel holder device, in particular a sample block device for accommodating at least one sample vessel and for holding said at least one sample. In this case, the sample vessel holder device preferably provides said first temperature section to which said at least one sample vessel and sample are thermally connectable, respectively.

Further, the apparatus preferably comprises at least one temperature device, which is adapted to cool and/or heat the sample. The temperature device, preferably, is the heat transport device. Further, the apparatus preferably comprises a heat sink device, which can form the second temperature section, wherein the at least one temperature device preferably is arranged between the sample block device and the heat sink device.

Preferably, the apparatus comprises a lid, which covers said at least one sample and which preferably provides said first temperature section. Moreover, it is possible that the apparatus provides a second lid, which is exchangeable with the first lid, wherein the solidification device is connected with the second lid, which, in this case, provides the first temperature section and a heat sink. The lid preferably comprises means for locking the lid with respect to the base body of the apparatus, which preferably holds the sample vessel holder, preferably also the heat transport means and preferably a heat sink. Preferably, the lid comprises means, e.g. mechanically, pneumatically or electrically driven means, for pressing the sample vessel(s) against the sample vessel holder device, e.g. against the thermoblock, for establishing tight thermal contact and for achieving a homogeneous temperature distribution over the thermoblock.

However, a problem can arise when pressing the sample vessels against the sample vessel holder device or generally, when accommodating the sample vessel(s) for a time period in a sample vessel holder device for performing any work step on the samples, e.g. the work step of measuring or thermally cycling the samples. The problem is that it can get difficult, e.g. by requiring a higher force, to remove the sample vessels from the sample vessel holder device, after they have been stored there for some time, e.g. under pressure. In particular robotic laboratory apparatus or fully automated laboratory apparatus, which comprise automatic lifts and other automated sample vessel transport devices, require that the removal of the sample vessels from a sample vessel holder device is smooth and reproducible in order to allow a failure-free processing of the samples. This also applies for manually operated devices. In this context, it was surprisingly observed by the inventors that the solidification by cooling the samples and sample vessels reliably assists in loosening the sample vessels from the sample vessel holder device. The reason is thought to be that the materials of the sample vessels filled with the non-solid sample, e.g. a microtiter plate made of plastics filled with an aqueous solution, and the material of the sample vessel holder device, e.g. a thermoblock made of aluminium, are different and have different thermal expansion coefficients. Cooling, or respectively freezing the vessels with the samples can lead to a spatial expansion of the vessel, which is higher than a possible spatial change of the well of the sample vessel holder device, which accommodates the vessel, thereby inducing mechanical stress between the components. In consequence, the sample vessels are loosened and slightly shifted out of the sample vessel holder device. This applies in particular, but not only, in the case that the wells of the sample holder device have a certain conical shape, i.e. if their diameter increases at least in a portion of the well towards the opening at the top side of the device, wherein the vessels basically have the same shape. This way, the removal of the sample vessels from the holder is facilitated.

Therefore, it is thought to be an invention to provide a method, using an apparatus, which further comprises a sample vessel holder device, which is configured to be the first temperature section and which has at least one well for accommodating at least one sample vessel, wherein the material forming the well has a thermal expansion coefficient, which is different from the thermal expansion coefficient of the sample vessel, and wherein the step of solidification of the at least one sample is performed such that the sample vessel is loosened within the well of the sample vessel holder device due to different thermal expansion of the materials, preferably by changing the relative positioning of the sample vessel relative to the sample block device is changed, preferably by thermally inducing mechanical stress between the sample block device and the at least one sample vessel, whereby the removal of the at least one sample vessel from the sample block device is facilitated after having performed the solidification step.

The solidification device preferably comprises temperature device, preferably a heat transport device for transporting heat between said first temperature section and said second temperature section, during a time period or a tempering period which starts at a start time. The heat transport device preferably comprises a resistive element, and/or a peltier element and/or a PTC-element, which allow a fast and precise adjustment of the first temperature by electrical control. In particular, peltier elements offer the advantage that they are easy to implement and control in laboratory apparatus. Further, the heat transport device can comprise a multistage pettier element. In a multistage peltier element, two or more peltier elements are combined in a sandwich-like setup to provide an improved performance of heat transport compared to single peltier elements. Multistage pettier elements are preferably used to cool down over a larger temperature difference, e.g. 50 K and more. A high performance of heat transport offers the advantage to reduce the time period of solidification. It is also possible and preferred that the heat transport device comprises additional or other temperature devices, e.g. based on the transport of a fluid to the first temperature section, the fluid serving to transport and exchange the heat. For example, a heat transport device could be utilized using the Joule-Thomson effect, by compressing and expanding a cooling medium, which flows in the vicinity of the samples, i.e. in the first temperature section, and which transports heat away.

The apparatus preferably comprises a second heat transport device, which can be the first heat transport device or an additional device and which provides an additional function. For example, such second heat transport device can be assigned to the second functionality, i.e. to said at least one work step, which can be the functionality of a thermocycler, wherein in this case the apparatus is adapted to provide the functions of a thermocycler.

The apparatus, in particular the solidification device, provides a control device for controlling the heat transport device, in particular to adjust the time period and the first temperature. This means, the control device is preferably adapted to at least influence the time period and the first temperature. Preferably, the control device is connected to the heat transport device, which adjusts the first temperature of the first temperature section during the time period. Preferably, the control device is adapted to control the solidification device by applying a predetermined time period and/or a first temperature, which are respectively appropriate for performing solidification of said at least one sample. Preferably, the apparatus comprises a second control device, which is adapted to control the solidification or to control other functions of the apparatus. The control device or the second control device, respectively, is an electrical device, i.e. it comprises electrical circuits, and can comprise a microcontroller or an FPGA, and can comprise calculation means, e.g. a microprocessor, data buses, power supply circuits, data input/output interfaces, data storage means and the like.

Preferably, the apparatus is adapted to cycle by means of said second heat transport device and said second control device the first temperature between at least two different cycle temperatures. This process called thermocycling is, for example, appropriate to perform a PCR on said at least one sample. According to a typical repetitive cycle of said thermocycling of a PCR, a sample of DNA molecules, in order to get copied or amplified respectively, with primers, which serve as start-DNA, and with nucleotides, which are attached to the primers, is heated to 95° C. within a first step of cycle, whereby the complementary strands of the DNA denature. By dropping the temperature to 55° C. within a second step of cycle, hybridization occurs, wherein the primers bind to the DNA. In a third step of cycle the sample is heated to 72° C. At this working temperature, the polymerase do assemble further nucleotides to the growing DNA strands and the loose bonds between primers and those DNA sections, which are not perfectly complementary, break open again. During the permanent repetition of a cycle, which is composed of said three cycle steps, the number of copied DNA molecules is doubled within each new cycle.

Preferably, the control device is adapted to determine the time period or tempering period, which is preferably reached by choosing a predetermined first temperature and a predetermined time period from a table of data, which might be stored in a data memory, which can be part of the control device or can be an additional device.

Moreover, it is possible and preferred, that the control device is adapted to determine the time period in dependence on a sample parameter. Such a sample parameter can be, for example, the first temperature. Preferably, the solidification device comprises at least one temperature sensor, which is adapted to measure the temperature of the first temperature section or the temperature of the sample or another characteristic temperature. Preferably, a low temperature value is set as the first temperature to perform the solidification. The time period depends on the samples material composition, the temperature at the beginning of the time period and the first temperature, which is the target temperature of the first temperature section at which solidification is performed.

It is preferred that the control device uses data, which are stored in a data memory inside the control device or outside the control device. Said data can comprise the settings for the time period in dependence on the start temperature, which is found at the first temperature section at the beginning of the time period, in dependence on the desired first temperature and in particular in dependence on the sample material. The choice of the first temperature, for example, in the case of a liquid sample depends on the sample solution, which may be based on water. Water changes its liquid state of matter to its solid state at 273.16 Kelvin at an ambient pressure of 611.73 Pa. However, other solvents usually have other melting points. In particular, the molarity of the solvent may influence the melting point. Long-chain alkanes, for example, have higher melting points. For example, Triacontane (C30H62) has a melting point of about 339 Kelvin. DMSO has a melting point of 292 Kelvin and Ethanol has a melting point of 158.75 Kelvin. Thus, in dependence on the melting point of the sample material, the first temperature is preferably in the range of 73.15 Kelvin to 243.15 Kelvin, is preferably in the range of 243.15 Kelvin to 264.14 Kelvin, is more preferably in the range of 248.15 K to 259.15 K, is preferably in the range of 264.15 Kelvin to 278.65 Kelvin and is preferably in the range of 278.65 to 353.15 Kelvin. Preferably, the first temperature is below 273.16 K and is preferably in the range of temperature 173 Kelvin to 273 K, preferably from 170 K to 264 K, preferably from 230 K to 256 K and most preferably from 250 K to 256 K. which has turned out in experiments to be an optimal value for the solidification of aqueous solutions, in particular for solutions containing PCR reagents. A first temperature of less than 264 K or less than 257 K offers the advantage that shorter time periods are necessary for the solidification.

Preferably, the first temperature is the set-temperature of a temperature controller with one or more closed loops, which can be implemented in the control device. The temperature sensors of the control loops are usually placed in contact with the sample vessel holder device. It should be noted that during the phase conversion of a non-solid sample, e.g. from liquid to solid, the temperature of the sample itself does not substantially change.

The time period for the solidification can be estimated by substantially using the known Planck's equation for freezing rates, which offers a mathematical relationship between the freezing time of a material in dependence on material parameters like density, molarity, heat capacity, sample thickness, the temperature of the first temperature section at the begin of the time period and the first temperature. Alternatively, the predetermined values of first temperature and time period can be determined experimentally and provided in said data memory as a table of data, which can also contain, in particular, the correction data, which regard the dependency of the time period and the first temperature on the sample material, the type of sample vessel and other relevant parameters. Generally, the lower the first temperature is, the shorter is the time period for the solidification of the sample. However, too low temperatures may damage certain sensible samples. Accordingly, the subsequent time periods experimentally turned out to be appropriate for the solidification of aqueous solutions, in particular for solutions containing PCR reagents: the time period is respectively preferably chosen from the ranges 0.15 to 0.8 minutes, 0.8 to 1.2 minutes, 1.2 to 1.5 minutes, 1.5 to 2.5 minutes, 2.5 to 5.0 minutes, 5.0 to 10.0 minutes or 10.0 to 15.0 minutes. However, other values of the first temperature and the time period are possible. The combination of a minimum time period between 25 seconds and 55 seconds and a first temperature between 240 K and 263 K (or between 250 K and 256 K) turned out to be appropriate for the solidification of aqueous samples, in particular PCR-samples, which are stored in a commercial plastic microtiter plate with 96 or 384 vessels, being accommodated in a metal thermoblock, which acts as the first tempering section.

Preferably, the solidification device comprises a solidification sensor, which measures a solidification parameter, which is appropriate to evaluate the solidification of said at least one sample. Preferably, the solidification device and/or the apparatus comprise a reference sample or a reference cell, on which the sensing of solidification is performed. Said reference may be arranged close to said at least one sample, e.g. in the thermoblock of a sample holder device. As solidification parameters, any physical, chemical or other parameters are appropriate, which depend on the state of matter such that a phase change in the state of matter, e.g. from liquid to solid, influences the solidification parameter. A solidification sensor can for example be based on a temperature sensor, which preferably is located in thermal contact with the sample, e.g. in contact with the sample vessel or which is surrounded by water in a cell or by another reference material. Herein, the temperature sensor measures the temperature-time curve of the reference material, which allows to derive the time-point of solidification of the reference material, e.g. by detecting a plateau-like characteristic of the temperature-time curve. Another preferred solidification sensor may, for example, use the interaction and, in particular the characteristic change of interaction, of light with the sample or reference sample upon solidification, e.g. the transmission, reflection, fluorescence, diffusion or refraction of light with the sample or the sample surface, respectively, as solidification parameters. In this case, a light source, e.g. a LED, and a light sensor, e.g. a photodiode, can be used to easily form a solidification sensor. Moreover, another preferred solidification sensor may comprise an acceleration, sound or vibration sensor, which is capable of detecting small changes and the vibrational characteristics of the sample or reference sample, which changes its vibrational characteristics when changing its state of matter. Preferably, such an acceleration sensor is mounted under an inelastic connection with the reference cell or a sample holder device, which holds the sample vessels with the samples. A particular embodiment of such a vibrational sensor may be based on the detuning of the resonance frequency of the sample or a reference cell upon solidification of the sample material or the reference cell. Further, a preferred solidification sensor may measure the electrical characteristics and its change upon solidification of a sample or a reference sample, e.g. by measurement of the electrical capacity or impedance. The control device may calculate an appropriate time period in dependence on parameters, e.g. the temperature, or the solidification parameter.

Preferably, the control device is adapted to determine the time period in dependence on said solidification parameter. In particular, the solidification device is adapted to stop tempering of the first temperature section, preferably even if the first temperature is not yet reached, if the solidification parameter indicates that said at least one sample has been transferred at least partially to a solid state of matter.

The apparatus and/or the solidification device further comprises a trigger device to trigger the solidification on said at least one sample at a start time. The trigger device is connected with the control device for data communication or may be integrated within the control device, preferably as a program code of a programmable control device. Preferably, the trigger device is implemented in the apparatus by using a program code as the trigger device, wherein the program code triggers the solidification in dependence on a predetermined event, e.g. a user interaction or any condition, which is automatically detected, e.g. the end of a work step, which was performed on the sample. It is preferred that the program code can be modified or generated by the user, via any user interface of the apparatus. It is further preferred that the program code is, at least in part, predetermined, and is run automatically. The program code, which triggers and controls the solidification can be stored in a non-volatile memory, e.g. a firmware. The trigger device is preferably adapted to be controlled manually or is preferably adapted to be controlled automatically. Preferably, the trigger device comprises a manual input device or an automatic input device, which may be connected wired or wireless to the trigger device. The trigger device gives control on the start time of the solidification of said at least one sample which allows, for example, that the apparatus first performs a work step on said at least one sample and afterward performs solidification. Most preferably, the solidification device and/or the apparatus is adapted such that, after initiating the solidification by means of the trigger device, i.e. triggering of the solidification, the solidification of said at least one sample takes place automatically.

Preferably the apparatus and/or the solidification device and/or the trigger device provide a manual input device, which may comprise a simple button or any other device, which is adapted to receive the action of a user to start the solidification. Further preferred, the apparatus and/or the solidification device and/or the trigger device provide an automatic input device, like an electronic data processing port or other communication interface, which is adapted to automatically receive and communicate a start signal, which is sent by another control device, computer or timer, to the control device, in order to start the solidification. The control device is preferably connected to said manual input device, which allows that the time period and the first temperature is influenced by the user, or may be further connected to another control device, which allows for the automatic adjustment of time period and the first temperature, or it is connected to additional devices, e.g. to sensors and/or actuators which measure/influence the time period and are the first temperature. Preferably, the apparatus and/or the solidification device is adapted such that besides a possible manual triggering no other user-actions, like programming or commanding operations, are required to let the apparatus and/or the solidification device perform the solidification of said at least one sample, which means that the apparatus and/or the solidification device preferably is prepared to perform solidification of said at least one sample automatically just by triggering the solidification. However, it is possible that the apparatus and/or the solidification device is adapted to require a conformational input by the user upon triggering the solidification, which may be a second push of the button after the first push, which triggers.

The apparatus further preferably comprises a feedback signal device which signals the progress and/or the completion of the solidification of said at least one sample. The feedback signal device can comprise an indication means which indicates the progress and/or the completion of the solidification of said at least one sample to the user, e.g. acoustically by sound, or visually, e.g. by an LED, which may emit blue light. Further, the feedback signal device may comprise a signal output means, e.g. a data communication port, which communicates the progress and/or the completion of the solidification of said at least one sample to an electronic data processing system by putting out an electronic feedback signal for further processing.

Preferably the apparatus according to the present invention comprises a reverse solidification device for reverting the solidification of said at least one sample, wherein by said reverse solidification said at least one sample is transferable at least partially to the state of matter, in which the sample was before the solidification, and wherein the solidification device comprises a third trigger device to trigger the reverse solidification of said at least one sample at a third start time, a third heat transport device for transporting heat between said first temperature section and said second temperature section during a third time period which starts at said third start time, and a third control device for controlling the third time period and the first temperature. The adjective “third” is generally used in the context of the present invention to refer to the reverse solidification device and its components and functions, if not stated contrary otherwise. Preferably, the reverse solidification device is prepared to perform reverse solidification of said at least one sample automatically just by triggering the reverse solidification.

The functionality of the reverse solidification device is substantially a reversion of the solidification. Reverse solidification means for example, that a partially frozen sample is transferred to a liquid state of matter, which preferably was the state of matter of the sample before the solidification. However, the sample may be transferred to a different state of matter upon reverse solidification. The advantage of the reverse solidification device is that it allows to control the time period, the schedule and the operation of the reversion of the solidification. Thus it is possible for example to perform at least one work step on said at least one sample, to solidify the sample in order to secure it against quality loss, and to reverse the solidification after a defined time period or after a manipulation of the solidified sample in order to receive the sample within a defined third time period in its state of matter, in which it was before the solidification. This allows a controlled work flow in a laboratory, in particular in an automated environment, and generally improves the period of laboratory work steps and the sample quality. For example, a liquid sample may be automatically frozen between work steps in a laboratory, which not only prevents leakage and contamination of the sample but also slows down chemical reactions in the sample and thus, prevents aging of the sample and conserves it respectively.

The reverse solidification device preferably comprises a set of subdevices and components, which respectively substantially correspond to the components of the solidification device and the respective functions. For example, the reverse solidification device respectively preferably comprises a third manual input device for manually controlling the third trigger device and/or a third automatic input device for automatically controlling the third trigger device. It is possible and preferred that the reverse solidification device shares one or more components or functional units with the solidification device. Respectively preferred, the third trigger device can be the trigger device, the third heat transport device can be the heat transport device, the third control device can be the control device, the third manual input device can be the manual input device, the third automatic input device can be the automatic input device and so on.

The third control device is preferably adapted to control the third time period and the first temperature. The third control device is preferably adapted to control the reverse solidification device by applying a third predetermined time period and/or a predetermined first temperature, which are appropriate for performing the reverse solidification of said at least one sample. Preferably, the control device is adapted to determine the time period and/or is preferably adapted to determine the time period in dependence on a sample parameter, preferably in dependence on the first temperature. Preferably, the reverse solidification device comprises a reverse solidification sensor which measures a reverse solidification parameter which is appropriate to evaluate the reverse solidification of said at least one sample and which can be the solidification sensor. Preferably, the control device is adapted to determine the time period in dependence on said reverse solidification parameter.

Preferably, the reverse solidification device comprises at least one third temperature sensor, which is adapted to measure the temperature of the first temperature section or the temperature of the sample. Also the temperature sensors may be shared used, i.e. for solidification and for reverse solidification. The first time period is preferably set to a first temperature in order to perform reverse solidification of said at least one sample, which is higher than the value of the first temperature which was set in order to perform the solidification of said at least one sample. Preferably, the first time period is preferably set to a first temperature in order to perform reverse solidification of said at least one sample, which is chosen from the ranges 253.15 K to 273.15 K, 273.15 K to 293.15 K, 293.15 K to 313.15 K, 313.15 K to 333.15 K or 333.15 K to 373.15 K. However, the first temperature for reverse solidification may be different. The predetermined third time period is respectively preferably chosen from the ranges 0.01 to 0.05 min, 0.05 to 0.15 min, 0.15 to 0.8 min, 0.8 to 1.2 min, 1.2 to 1.5 min, 1.5 to 2.5 min, 2.5 to 5.0 min, 5.0 to 10.0 min or 10.0 to 15.0 min. However, the predetermined third time period may be different.

The reverse solidification device is preferably adapted to be arranged in an apparatus according to the present invention. Preferably the reverse solidification is a module of the apparatus according to the present invention. Further it is possible and preferred that the reverse solidification device shares most components and functions with the solidification device, and preferably shares the same housing with the solidification device. In this case, the functionality of reverting the solidification of said at least one sample is preferably implemented in the solidification device, e.g. by using a programmable control device, which is run with operational data or programs, which respectively are adapted to perform reverse solidification of said at least one sample by means of said programmable control device. Such an embodiment may be used for example in combination with a solidification device, which is adapted as standalone device. In this case, said at least one sample, which may be provided in a 96 sample tray and may be placed in an apparatus according to the present invention, may be removed from said apparatus in order to perform a reverse solidification separate and independent from the apparatus, by means of the stand-alone solidification device.

Referring to the explanations, definitions related to the apparatus and the solidification device, it is a further aspect of the present invention to provide a method for the processing of at least one non-solid sample, using a laboratory apparatus, which is designed for performing a work step on said sample, wherein the apparatus comprises a solidification device for the solidification of said sample and at least one triggering device for triggering the solidification, the method comprising the steps:—providing at least one non-solid sample in a laboratory apparatus;—triggering the solidification of said at least one non-solid sample by means of the triggering device; and performing the solidification by means of the solidification device.

It is preferred for the method that the laboratory apparatus further comprises at least one first temperature section, at least one second temperature section and at least one heat transport device for transporting heat between the first and second tempering section, wherein the at least one non-solid sample is thermally connected to said at least one first tempering section and the at least one heat transport device is assigned to the solidification device, and wherein the step of solidification of the at least one non-solid sample is performed by transporting heat out of the at least one non-solid sample during a first time period such that the sample is at least in part transferred to a solid state, e.g. by freezing, or that the viscosity of the sample is increased.

It is further preferred for the method that the laboratory apparatus further comprises an electrical control device, and wherein the method further comprises the steps of controlling the solidification device by means of the control device by applying a first temperature to the first temperature section during a first time period, wherein the combination of parameters of the first temperature and the first time period are appropriate for performing the solidification of said at least one sample.

It is further preferred for the method that the laboratory apparatus further comprises at least one temperature sensor for measuring the first temperature and at least one electrical closed loop for adjusting the first temperature, wherein the method further comprises the step of adjusting the first temperature section to the first temperature.

The step of solidification is performed preferably after said work step of said apparatus. In consequence, the at least one sample is secured against leakage, whereby subsequent handling of the sample is simplified and an improvement of sample quality is achieved.

Method-like features, which are related to the above described features of the apparatus and the solidification device, e.g. manually and/or automatically triggering the solidification device by means of the manual and/or automatic input device, may be adapted by the skilled person to the method according to the present invention, and repetition is therefore omitted.

Using the explanations and definitions of the description of the present invention, in particular the following embodiments of the apparatus, the solidification device and the method according to the present invention are provided:

The apparatus according to the present invention, in particular for use in a laboratory, for performing a work step on at least one sample, comprising a first temperature section, which is adjustable to have a first temperature and to which said at least one sample is thermally connectable, a second temperature section with a second temperature, and a solidification device for the solidification of said at least one sample, wherein by said solidification said at least one sample is transferable at least partially to a solid state of matter, and wherein the solidification device comprises a trigger device to trigger the solidification of said at least one sample at a start time, a heat transport device for transporting heat between said first temperature section and said second temperature section during a time period which starts at said start time, and a control device for controlling the time period and the first temperature.

The apparatus according to the present invention which is prepared to perform solidification of said at least one sample automatically just by triggering the solidification.

The apparatus according to the present invention characterized in that it comprises a manual input device for manually controlling the trigger device.

The apparatus according to the present invention characterized in that it comprises an automatic input device for automatically controlling the trigger device.

The apparatus according to the present invention characterized in that the control device is adapted to control the time period and the first temperature.

The apparatus according to the present invention characterized in that the control device is adapted to control the solidification device by applying a predetermined time period and/or a predetermined first temperature, which are respectively appropriate for performing solidification of said at least one sample.

The apparatus according to the present invention characterized in that it comprises a sample holder device for holding said at least one sample which provides said first temperature section.

The apparatus according to the present invention characterized in that it comprises a thermoblock which provides said first temperature section.

The apparatus according to the present invention characterized in that it comprises a lid which covers said at least one sample and which provides said first temperature section.

The apparatus according to the present invention characterized in that it comprises a feedback signal device which signals completion of the solidification of said at least one sample.

The apparatus according to the present invention characterized in that the feedback signal device comprises an indication means which indicates completion of the solidification of said at least one sample to the user.

The apparatus according to the present invention characterized in that the feedback signal device comprises a signal output means which communicates the completion of the solidification of said at least one sample to an electronic data processing system.

The apparatus according to the present invention characterized in that it comprises a second heat transport device for transporting heat during a second time period.

The apparatus according to the present invention characterized in that the heat transport device and/or the second heat transport device comprise a pettier element.

The apparatus according to the present invention characterized in that the heat transport device and/or the second heat transport device comprises a multistage pettier element.

The apparatus according to the present invention characterized in that the heat transport device and the second heat transport device are the same device.

The apparatus according to the present invention characterized in that it comprises a second control device which is adapted to control said second heat transport device.

The apparatus according to the present invention characterized in that the control device and the second control device are the same device.

The apparatus according to the present invention characterized in that it is adapted to cycle by means of said second heat transport device and said second control device the first temperature between at least two different cycle temperatures.

The apparatus according to the present invention characterized in that it comprises the substantial functions of a thermocycler or is a thermocycler and that said work step is the process of thermocycling.

The apparatus according to the present invention characterized in that said at least one sample contains reagents for a polymerase chain reaction (PCR) and that the apparatus is adapted to perform a PCR on said at least one sample.

The apparatus according to the present invention characterized in that it comprises the substantial functions of a thermostat or is a thermostat and that said work step is the process of setting said at least one sample to a target temperature.

The apparatus according to the present invention characterized in that said work step is to hold said at least one sample for later use.

The apparatus according to the present invention characterized in that said work step is the solidification of at least one sample.

The apparatus according to the present invention characterized in that it comprises a plurality of solidification devices.

The apparatus according to the present invention being a thermocycler, i.e. a thermocycler for thermocycling at least one sample, comprising a first temperature section, which is adjustable to have a first temperature and to which said at least one sample is thermally connectable, and a second temperature section with a second temperature, comprising a solidification device for the solidification of said at least one sample, wherein said at least one sample is transferable at least partially to a solid state of matter such that leakage of the sample is prevented, and wherein the solidification device comprises a trigger device to trigger the solidification of said at least one sample at a start time, a heat transport device for transporting heat between said first temperature section and said second temperature section during a time period which starts at said start time, and a control device for controlling the heat transport device.

The apparatus and/or the control device are preferably configured for running the method according to the invention, in particular by providing appropriate heat transporting means, which are capable to apply the first temperature to the first temperature section and/or by providing a memory, which stores data, in particular program code or a table with physical parameters time period and first temperature, which are required for running the method according to the invention, in particular for triggering and performing the solidification.

Solidification device according to the present invention for the arrangement in an apparatus, for the solidification of at least one sample, wherein by said solidification said at least one sample is transferred at least partially to a solid state of matter such that leakage of the sample is prevented, wherein the solidification device comprises a trigger device to trigger the solidification of said at least one sample at a start time, a heat transport device for transporting heat between a first temperature section, which is adjustable to have a first temperature and to which said at least one sample is thermally connectable, and a second temperature section during a time period, which starts at a start time, and a control device for controlling the heat transport device.

Solidification device according to the present invention characterized in that it is prepared to perform solidification of said at least one sample automatically just by triggering the solidification.

Solidification device according to the present invention characterized in that it comprises a manual input device for manually controlling the trigger device.

Solidification device according to the present invention characterized in that it comprises an automatic input device for automatically controlling the trigger device.

Solidification device according to the present invention characterized in that the control device is adapted to control the time period and the first temperature.

Solidification device according to the present invention characterized in that the control device is adapted to control the solidification device by applying a predetermined time and/or a predetermined first temperature, which are appropriate for performing solidification of said at least one sample.

Solidification device according to the present invention characterized in that the control device is adapted to determine the time period.

Solidification device according to the present invention characterized in that the control device is adapted to determine the time period in dependence on a sample parameter.

Solidification device according to the present invention characterized in that the control device is adapted to determine the time period in dependence on the first temperature.

Solidification device according to the present invention characterized in that it comprises a solidification sensor which measures a solidification parameter which is appropriate to evaluate the solidification of said at least one sample.

Solidification device according to the present invention characterized in that the control device is adapted to determine the time period in dependence on said solidification parameter.

Solidification device according to the present invention characterized in that it comprises a temperature sensor which is adapted to measure the temperature of the first temperature section or the temperature of the sample.

Solidification device according to the present invention characterized in that the first temperature is preferably in the range of 73.15 K to 243.15 K, is preferably in the range of 243.15 K to 264.14 K, is more preferably in the range of 248.15 K to 259.15 K, is preferably in the range of 264.15 K to 278.65 K and is preferably in the range of 278.65 K to 353.15 K.

Solidification device according to the present invention characterized in that the predetermined time period is respectively preferably chosen from the ranges 0.01 to 0.05 min, 0.05 to 0.15 min, 0.15 to 0.8 min, 0.8 to 1.2 min, 1.2 to 1.5 min, 1.5 to 2.5 min, 2.5 to 5.0 min, 5.0 to 10.0 min or 10.0 to 15.0 min.

Solidification device according to the present invention for the arrangement in an apparatus according to the present invention.

Solidification device according to the present invention for the arrangement as a module of the apparatus according to the present invention.

Method according to the present invention for performing the solidification of at least one sample in an laboratory apparatus, which is adapted to perform a work step, comprising the steps: providing a first temperature section which is adjustable to have a first temperature and providing a second temperature section with a second temperature; connecting said at least one sample thermally to said first temperature section, triggering the solidification at a start time by means of a trigger device, and performing solidification of said at least one sample by transferring it at least partially to a solid state of matter by means of a solidification device such that leakage of the sample is prevented.

Method according to the present invention characterized in that it comprises a step of transporting heat by means of a heat transport device between said first temperature section and said second temperature section during a time period which starts at said start time.

Method according to the present invention characterized in that it comprises a step of controlling said heat transport device by means of a control device.

Method according to the present invention characterized in that it comprises a step of controlling said start time and said first temperature by means of a control device.

Method according to the present invention characterized in that it comprises a step of controlling the time period by means of said control device.

Method according to the present invention characterized in that the step of triggering is controlled manually.

Method according to the present invention characterized in that the step of triggering is controlled automatically.

Method according to the present invention characterized in that said work step comprises thermocycling of said at least one sample which includes cycling of said first temperature between at least two different cycle temperatures.

Method according to the present invention characterized in that said solidification is performed after said work step of said apparatus.

Method according to the invention, wherein the laboratory apparatus is a thermocycler, wherein the non-solid sample is an aqueous solution with PCR-reagents and wherein the work step comprises the repetitive thermal cycling of the at least one sample.

Method according to the invention, wherein the apparatus further comprises a sample block device, which is configured to be the first temperature section and which has at least one well for accommodating at least one sample vessel, wherein the material forming the well has a thermal expansion coefficient, which is different from the thermal expansion coefficient of the sample vessel, and wherein the step of solidification of the at least one sample is performed such that the sample vessel is loosened within the well of the sample vessel holder device due to different thermal expansion of the materials

Method according to the invention, wherein the apparatus further comprises means for removing at least one sample vessel, in particular a microwell plate or other multi-sample-vessels, from the sample vessel holder device, which can be a thermoblock of a thermocycler. The method, preferably, provides the step to remove the at least one sample vessel from the sample holder device of the apparatus, wherein said step is preferably performed after the at least one sample vessel has been loosened within the at least one well of the sample vessel holder device by the solidification of the sample.

The means for removing at least one sample vessel can comprise a moving element at the apparatus or associated to the apparatus, which is arranged and configured to move the at least one sample vessel, in particular to lift and/or transport the at least one sample vessel. The movable element can be adapted to be part of a grappler device or a hook device or holding arm, for example, and can be connected to a stand device or socket, which carries the movable element, and which can be part of the apparatus or separate, but associated to the apparatus. The movable element can be part of a robot system for transporting sample vessels, which further can be part of a laboratory management system (LMS) or a laboratory information management system (LIMS), respectively, which are software-based laboratory and information management systems with automated operation of samples, including, e.g., sample registration, sample transport, and stations for performing at least one work step on a sample, e.g. sample analysis or a thermocycler performing a PCR-process.

Preferably, the apparatus is configured to automatically remove the at least one sample vessel from the sample vessel holder device. This can be achieved, for example, by providing a programmed electric control device with a program code, which controls the operation of the means for removing at least one sample vessel. The program code preferably is adapted to let the movable element remove the at least one sample vessel from the sample vessel holder, in particular after the solidification of the sample. In a further aspect, the sample vessel holder device can be heated for a predetermined period before removing the vessel such that a thin film of frozen condense water is unfrozen, which e.g. may connect the sample vessel and the sample vessel holder device, in particular after the solidification by freezing, but such that the sample substantially remains solidified.

It is preferred to use the apparatus according to the present invention or a solidification device according to the present invention for solidification of at least one sample.

It is preferred to use the apparatus according to the present invention or a solidification device according to the present invention for performing the method according to the present invention.

It is preferred to use the apparatus for performing a work step on at least one sample, comprising a first temperature section, which is adjustable to have a first temperature and to which said at least one sample is thermally connectable, a second temperature section with a second temperature, a heat transport device for transporting heat between said first temperature section and said second temperature section during a time period which starts at said start time, and a control device for controlling the time period and the first temperature, to perform the solidification of said at least one sample, wherein by said solidification said at least one sample is transferable at least partially to a solid state of matter such that leakage of the sample is prevented.

It is preferred to use a commercial thermocycler for thermocycling at least one sample, to perform the solidification of said at least one sample, wherein by said solidification said at least one sample is transferable at least partially to a solid state of matter such that leakage of the sample is prevented.

It is preferred to use a commercial PCR-instrument for performing a PCR reaction on at least one sample, to perform the solidification of said at least one sample, wherein by said solidification said at least one sample is transferable at least partially to a solid state of matter such that leakage of the sample is prevented.

A program code is preferably also part of the invention which controls the operation of the apparatus according to the present invention, the solidification device according to the present invention, the reverse solidification device according to the present invention and/or or the method of performing the solidification of at least one sample according to the present invention.

A date storage medium is preferably also part of the invention which contains data for operating the apparatus according to the present invention, the reverse solidification device according to the present invention and/or the solidification device according to the present invention, and/or for performing the method according to the present invention and/or for use with the programming code according to the present invention.

Further advantages, features and applications of the present invention can be derived from the following embodiments of the apparatus and the solidification device and the methods according to the present invention with reference to the drawings. Further, the apparatus and the solidification device according to the present invention may have different parts and functions, which for reason of simplicity are not shown in the figures. Equal reference signs in the figures substantially describe equal devices.

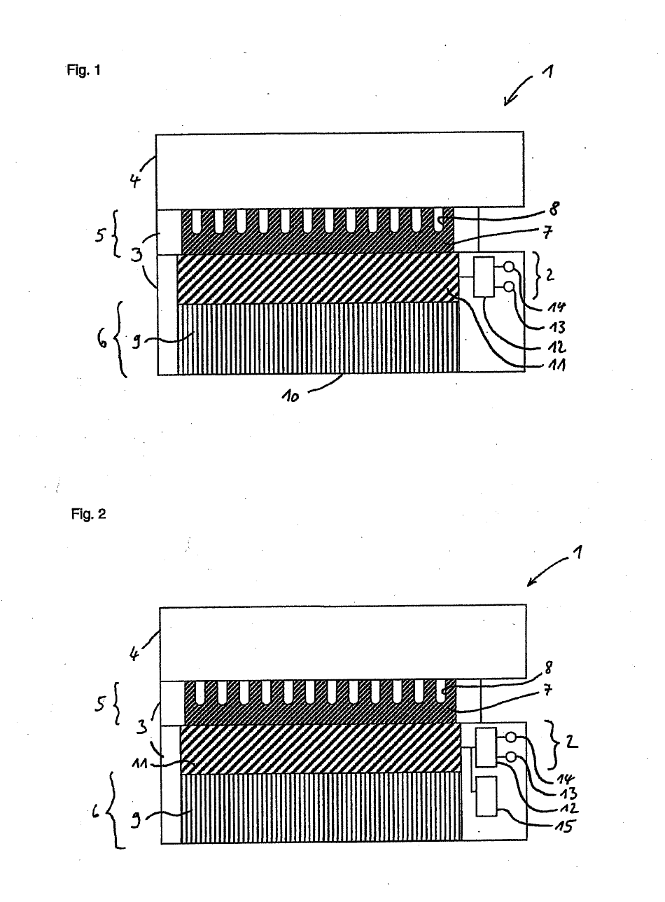

FIG. 1 shows a first example of the apparatus according to the present invention with a solidification device according to the present invention;

FIG. 2 shows second example of the apparatus according to the present invention with a solidification device according to the present invention;

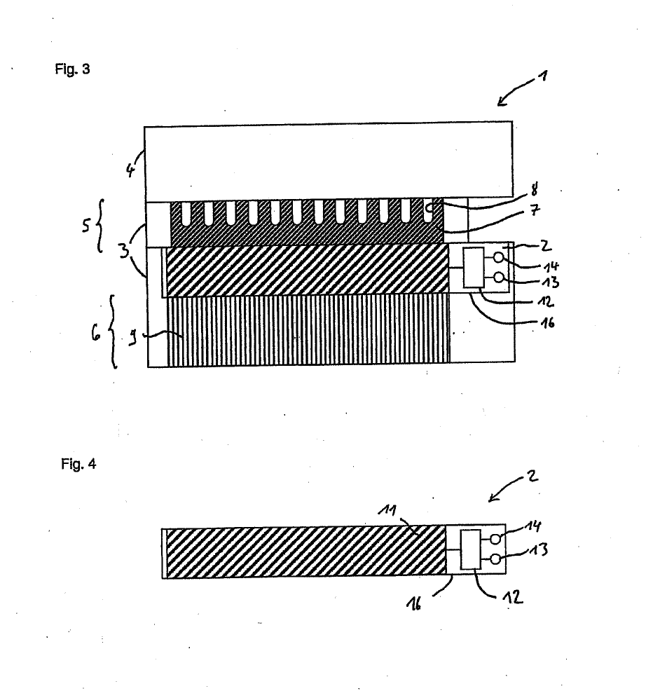

FIG. 3 shows a third example of the apparatus according to the present invention with a solidification device according to the present invention;

FIG. 4 shows a first example of the solidification device according to the present invention;

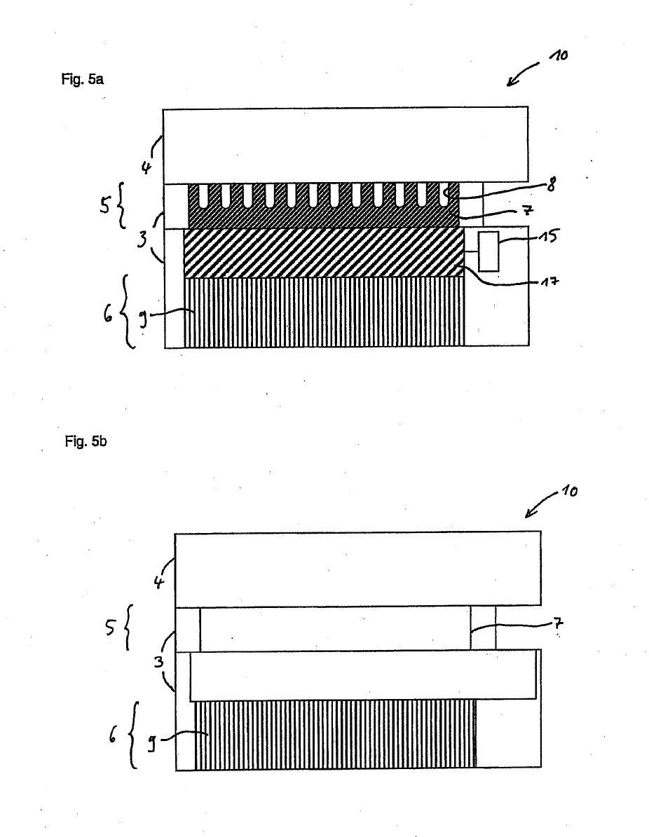

FIG. 5a shows an apparatus which is capable of being a retrofitted with a solidification device according to the present invention;

FIG. 5b shows the apparatus according to FIG. 5, which is being prepared for being a retrofitted with a solidification device according to the present invention;

FIG. 5c shows the apparatus according to FIG. 5 which is retrofitted with a solidification device according to the present invention, thus representing an apparatus according to the present invention;

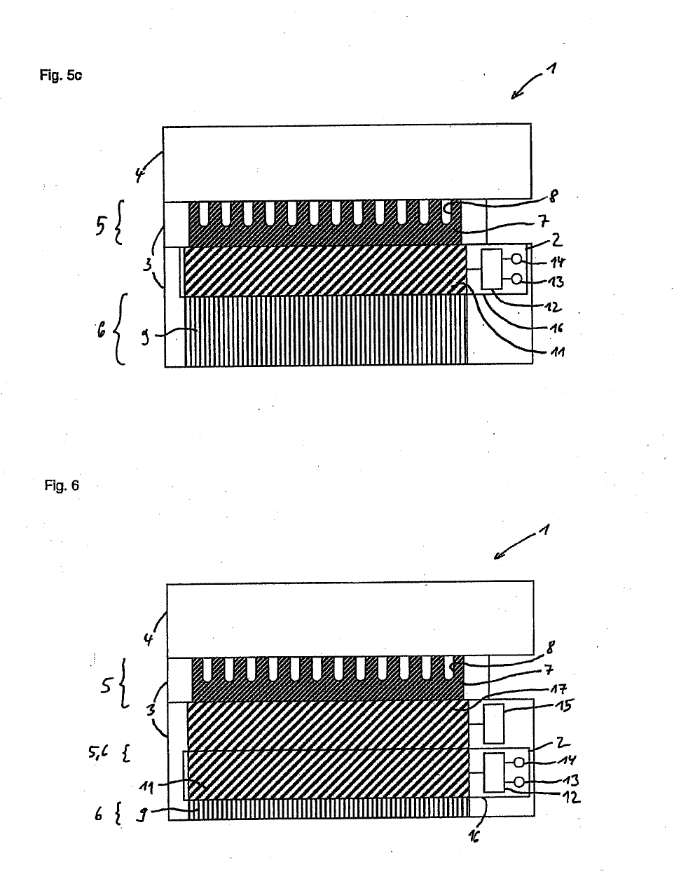

FIG. 6 shows a fourth example of the apparatus according to the present invention with a solidification device according to the present invention;

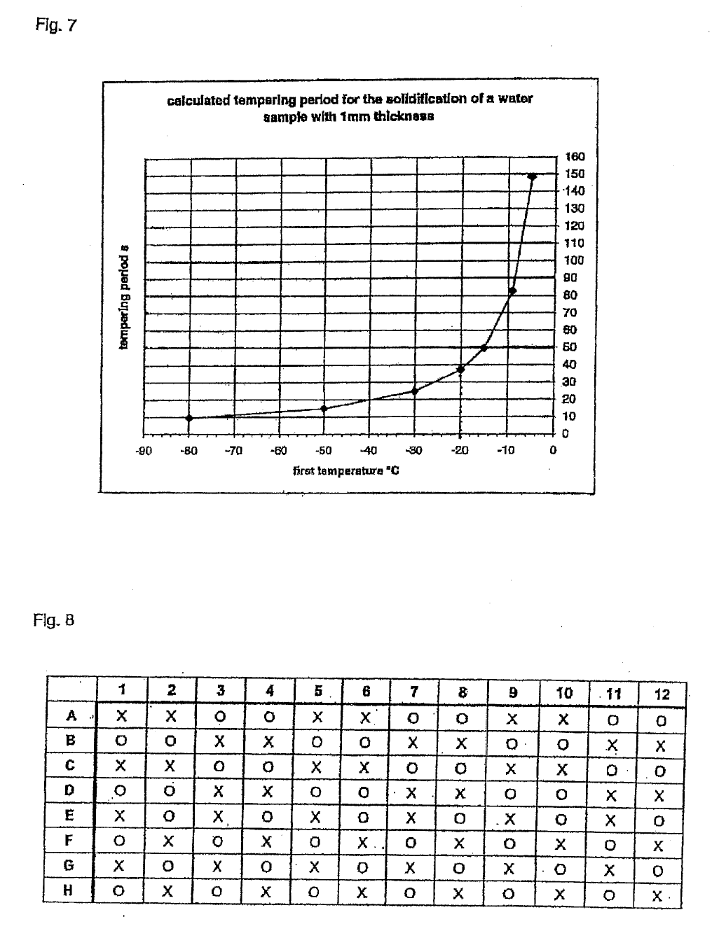

FIG. 7 shows a diagram with a graph which represents the calculated tempering period for the complete solidification of a water sample with one millimeter thickness;

FIG. 8 shows a table, which illustrates the layout of a 96 PCR plate for testing potential cross contamination when using a method according to the present invention.

FIG. 1 shows a first example of the apparatus 1 according to the present invention with a solidification device according to the present invention. In the example of the apparatus according to the present invention in FIG. 1, the solidification device and its functional components are integrated within the apparatus 1. The apparatus 1 comprises a carrier part 3, which carries several functional components including the solidification device 2, and further comprises the lid part 4, which covers the samples. The apparatus further has a first temperature section 5, which is in particular formed by a sample holder device 7. The first temperature section can be adjusted to have a first temperature, at which solidification can be performed on the samples. The apparatus further comprises a second temperature section 6, which basically consist of a heat sink 9, which on its lower side 10 is thermally connected with the environment of the apparatus. The solidification device 2 comprises the components of a heat transport device 11, a control device 12 and a trigger device 13.

The sample holder device 7 is a thermoblock made of silver, which comprises 96 openings 8 arranged in an array. The thermoblock and said array are adapted to receive standard industrial 96 PCR-plates. Thus, an optimal thermal connectivity between the sample vessels (not shown) of the PCR-plate and the thermoblock is provided. The sample vessels each contain a sample, which is an aqueous solution with PCR-reagents. The openings of the sample vessels are sealed with a heat sealing film (not shown).

The heat transport device 11 of the solidification device in FIG. 1 implements two basic functionalities in the example of the apparatus according to the present invention. The first functionality is the solidification of the samples. The second function of the heat transport device 11 in the example of FIG. 1 is the functionality of a thermocycler. The heat transport device of the thermocycler cycles the temperature of the thermoblock 7 between at least two different temperatures, wherein the cycle time schedule, the tempering periods and tempering rates are predetermined and controlled by the control device 12. The apparatus according to the example in FIG. 1 is therefore adapted to fulfil the functions of a thermocycler. This applies also for the other examples of the apparatus in FIGS. 2, 3, 5 and 6. The purpose of the thermocycler is to perform a polymerase chain reaction (PCR) on the samples in the sample vessels 8, which contain an aqueous solution with PCR reagents, which will perform the amplification reaction of the PCR upon thermocycling. The heat transport device 11 comprises a peltier unit with several peltier elements arranged in parallel, not stacked on top of each other but arranged in one layer, which electrically driven transport heat from the first temperature section 5 to the second temperature section 6. The heat sink 9 in the second temperature section 6 is a copper block with fins, which form a large contact surface between the heat sink 9 and the environment, to improve the heat exchange. Since the figures are only schematically, the dimension of the components in the figures do not represent the real dimensions. In particular, the dimension of the heat sink in the vertical direction (perpendicular to the side 10) is preferable substantially larger than the dimension of the pettier element of the device 11 in the same direction.

In FIG. 1, the control device 12 is a microcontroller. Control device 12 integrates, like the heat transport device 11, at least two different functionalities. First, it controls the functionality of the solidification device, which includes the control of the solidification operation which is performed to solidify the samples. Second, the functionality of the thermocycler is controlled. Said integration of functionalities is realized in the example of FIG. 1 by a program control of the control device 12. The control device 12 controls the functionalities by running at least one specific program, which is chosen by a user and/or automatically chosen. Said program uses program data and operational data. Said data are predetermined for certain standard applications of the apparatus and are contained in a memory of the control device 12, where they can be chosen by the user, by the control device or by data exchange with an external PC, which is connectable to the apparatus 1 and to the control device 12.

The control device 12 comprises control circuits (not shown) which control the first temperature and the cycle temperatures by using temperature data, which are measured by temperature sensors (not shown) arranged in the thermoblock 7.

In FIG. 1, the solidification device comprises the trigger device 13, in order to trigger the solidification of the samples. In the example, the trigger device 13 comprises a manual input device, which is a button arranged on the outside of the case of the carrier part 3 of the apparatus, which allows the user to manually initiate the solidification of the samples. Upon pushing the button, the samples are automatically solidified, if no other program is running. However, the trigger device 13 also comprises an automatic input device (not shown), which is realized by means of the programmable circuitry of the control device 12. The automatic input device comprises the capability of the program, which is run by the control device 12 for controlling the functionalities, to automatically initiate the solidification. The automatic input device initiates the solidification by calling another program or program subroutine, which is adapted to control the solidification functionality of the heat transport device 11. The thermocycler 1 of FIG. 1 is adapted by means of the control device 12 to perform solidification of the samples automatically after the termination of the PCR. After the program, which controls the thermocycling, ends, the subprogram, which controls the solidification, is automatically called. This way, the triggering of the solidification is implemented automatically. The end time of the solidification can be determined by a timer, timer subprogram or be determined by the detection of the solidification by means of a solidification sensor. In the example, a timer subroutine of the program, which is run by the control device 12, communicates the end of the solidification to a feedback signal device (not shown). It is preferred that the apparatus has a user interface, e.g. a display with a keyboard or a touch-screen, which is provided for letting the user modify a program, which controls the first and second functionality of the apparatus, namely the thermal cycling of the samples and the subsequent solidification of the samples. It is preferred that the user just decides if or not the solidification is required and, preferably, the apparatus automatically chooses and, if so, also displays, the parameters of the time period and first temperature of the solidification. However it is also preferred that the user can determine at least one of both parameters, e.g. in order to choose a first temperature, which will not damage the sample, while the apparatus then preferably chooses the appropriate time period automatically.

The feedback signal device, realized as another part of the program, signals the completion of the solidification of said at least one sample to the user. This is achieved by an indication means 14, which is part of the feedback signal device, which indicates completion of the solidification of said at least one sample to the user. Said indication means 14 is e.g. a blue light emitting diode. After completion of the solidification, the PCR-plate may be removed from the apparatus and the sealing film may be removed from the PCR-plate without the risk of spreading the sample solution. Thus, the risk of cross-contamination of the samples is reduced.

FIG. 2 shows second example of the apparatus according to the present invention with a solidification device according to the present invention. The apparatus of FIG. 2 differs from the apparatus of FIG. 1 by providing a second control device 15, which is a microcontroller. In FIG. 2, the second control device controls the functionality of the thermocycler while the (first) control device controls the solidification device.

FIG. 3 shows a third example of the apparatus according to the present invention with a solidification device 2 according to the present invention. The solidification device 2 of this apparatus is formed as a module according to FIG. 4 and comprises a case 16, the heat transport device 11, the control device 12, the trigger device 13 and the indication means 14.

The modular solidification device 2 of FIG. 4 is preferably used to retrofit an apparatus 10, e.g. a thermocycler 10, which is not adapted to perform the solidification of a sample, with a solidification device, e.g. the solidification device of FIG. 4.

The thermocycler 10 of FIG. 5a comprises the heat transport device 17 and the control device 15, which is adapted to control the functionality of the thermocycler. However, the heat transport device 17 and the control device 15 are not appropriate and not adapted to perform solidification of a sample. Therefore, in FIG. 5b, the heat transport device 17, and the control device 15—and alternatively also the sample holder device 7—are removed and replaced by the solidification device 2, which means that the apparatus 10 is retrofitted by a solidification device 2, yielding an apparatus 1 according to the present invention (FIG. 5c). The solidification device 2 of FIG. 5c is constructed according to the example of FIG. 4. In FIG. 5, the control device 12 controls both functionalities of the apparatus 1, the solidification and the thermocycling.

Instead of replacing the heat transport device 17 and the control device 15, the solidification device 2 may be additionally provided to the apparatus 10. Thus, in FIG. 6, the solidification functionality is added to the thermocycler 10 of FIG. 5a by adding the solidification device 2. The constructive solution may require either a stacking or parallel arrangement of the peltier elements of the heat transport device 11 and 17.

FIG. 7 shows a diagram with a graph which represents the calculated tempering period for the complete solidification of a water sample with one millimeter thickness. The calculation is based on standard material constants for water and uses the usual Planck Equation for freezing rate prediction. The calculated tempering period is used to estimate values for the tempering period, which may be appropriate to perform the solidification of samples containing an aqueous solution of PCR-reagents. Said values can be used to provide operational data for operating the apparatus and the solidification device according to the present invention, in particular the apparatus 1 and the solidification device 2 according to the drawings. The control device 2 or an external device may be adapted to calculate such values for use as operational data during operation of the apparatus 1 or the solidification device 2. However, said values may also be predetermined by calculating them externally or by experimental measurement, in order to supply them afterwards as operational data to the apparatus 1, the solidification device 2 and the control device 12 respectively. As an example of operational data, the first temperature may be 253.15 K, i.e. −20° C., which leads to an estimated value for the tempering period of 37 seconds, which is appropriate to solidify an aqueous sample by the solidification device 2.

FIG. 8 shows a table, which illustrates the layout of a 96 PCR plate for testing potential cross contamination when using a method according to the present invention. The test, which was performed and is illustrated here, demonstrates the advantage of the apparatus, the solidification device and the method according to the present invention. In summary, no cross-contamination was detected in any of the 96 samples upon mechanical manipulation of the samples, which were solidified before the manipulation by a solidification device according to the present invention in an apparatus according to the present invention.

A 96 PCR-plate was filled with two different types of PCR-samples, both types of samples containing a different PCR-reagent, for a different PCR-reaction each. The table of FIG. 8 shows the layout of sample fill of the 96 PCR plate, wherein the first type of PCR-sample is labelled with “x” and the second type is labelled with “o”. The samples “x” are related to a PCR system of Actin gene from hg-DNA and the samples “o” are related to a PCR system of GFP gene from Plasmid-DNA. Both PCR-reactions take place under the same conditions.

The samples were filled in the sample vessels of the 96 PCR-plate from type “Eppendorf twin.tec 96”, the PCR-plate was sealed by a heat sealing film and centrifuged for 1 min at 1000 rpm. The PCR-plate was then transferred to an apparatus according to the present invention, which here is a thermocycler with solidification device. First, PCR-reactions were performed by running 40 thermocycles. After termination of the PCR-reactions, solidification of the samples was performed at 253.15 K for 0.75 min, whereby the samples were partially frozen. The PCR-plate was removed manually from the apparatus. Without further centrifugation, the PCR-film was manually removed from the PCR-plate, the samples were allowed to unfreeze under room temperature and the sample solutions were taken and analyzed each by agarose gel electrophoresis. Usually, the same procedure would, without performing the solidification by the solidification device, lead to the detection of some cross-contamination by the electrophoresis test. At hand, no cross-contamination was detected in any of the samples by using the step of solidification by the solidification device. This proofs that the apparatus, the solidification device and the method according to the present invention are appropriate to improve the manageability of laboratory samples, thereby improving their stability against leakage and thus, enhancing the reliability of sample processing.

1. Method for the processing of at least one non-solid sample, using a laboratory apparatus (1; 10), which is designed for performing a work step on said sample, wherein the apparatus comprises a solidification device (2) for the solidification of said sample and at least one triggering device (13) for triggering the solidification, the method comprising the steps:
providing at least one non-solid sample in a laboratory apparatus,
triggering the solidification of said at least one non-solid sample by means of the triggering device and
performing the solidification by means of the solidification device.

2. Method according to claim 1 wherein
the laboratory apparatus further comprises at least one first temperature section (5), at least one second temperature section (6) and at least one heat transport device (11) for transporting heat between the first and second tempering section, wherein the at least one non-solid sample is thermally connected to said at least one first tempering section and the at least one heat transport device is assigned to the solidification device, and
wherein the step of solidification of the at least one non-solid sample is performed by transporting heat out of the at least one non-solid sample during a first time period such that the sample is at least in part transferred to a solid state, e.g. by freezing, or that the viscosity of the sample is increased.

3. Method according to claim 2 wherein
the laboratory apparatus further comprises an electrical control device (12), and
wherein the method further comprises the steps of
controlling the solidification device by means of the control device by applying a first temperature to the first temperature section during a first time period, wherein the combination of parameters of the first temperature and the first time period are appropriate for performing the solidification of said at least one sample.

4. Method according to claim 3 wherein
the laboratory apparatus further comprises at least one temperature sensor for measuring the first temperature and at least one electrical closed loop for adjusting the first temperature,
wherein the method further comprises the step of adjusting the first temperature section to the first temperature.

5. Method according to claim 3 or 4 wherein the first temperature is predetermined and chosen from the range of temperature 173 Kelvin to 273 K, preferably from 170 K to 264 K, preferably from 230 K to 256 K and most preferably from 250 K to 256 K.

6. Method according to claim 3, 4 or 5 wherein the first temperature is predetermined and respectively preferably chosen from the time ranges 0.05 to 0.15 min, 0.15 to 0.8 min, 0.8 to 1.2 min, 1.2 to 1.5 min, 1.5 to 2.5 min, 2.5 to 5.0 min, 5.0 to 10.0 min or 10.0 to 15.0 min.

7. Method according to any of the previous claims, further comprising the step:
performing the step of solidification after having performed any work step on said at least one sample, said work step being different from the step of solidification.

8. Method according to any of the previous claims, further comprising the steps:
storing the at least one non-solid sample in at least one sample vessel, which is closed by means of at least one means for closing and which is arranged in the laboratory apparatus;
performing the step of solidification of the at least one sample prior to a step of opening at least one of the sample vessels or prior to a step of transporting the at least one sample vessel.

9. Method according to any of the previous claims 2 to 8, wherein
the apparatus further comprises a sample vessel holder device, which is configured to be the first temperature section and which has at least one well for accommodating at least one sample vessel, wherein the material forming the well has a thermal expansion coefficient, which is different from the thermal expansion coefficient of the sample vessel, and
wherein the step of solidification of the at least one sample is performed such that the sample vessel is loosened within the well of the sample vessel holder device due to different thermal expansion of the materials.

10. Method according to any of the previous claims, the apparatus further comprising a sample vessel holder device for holding at least one sample, the method further providing the step of removing, in particular automatically, the at least one sample vessel from the sample holder device of the apparatus, in particular after performing the steps of claim 9.

11. Laboratory apparatus (1; 10) for performing a work step on at least one sample,
comprising a first temperature section (5), which is adjustable to have a first temperature and to which said at least one sample is thermally connectable,
a second temperature section (6) with a second temperature, and
a solidification device (2) for the solidification of at least one non-solid sample, wherein by said solidification said at least one non-solid sample is transferable at least partially to a solid state of matter or to a more viscous state of matter,
wherein the solidification device comprises
a trigger device (13) to trigger the solidification of said at least one non-solid sample at a start time,
a heat transport device (11) for transporting heat between said first tempering section and said second tempering section during a time period which starts at said start time, and
an electrical control device (12) for controlling the solidification by controlling the application of the time period and the first temperature.

12. Laboratory apparatus according to claim 11, wherein the control device is programmable and configured to run a predetermined program for the solidification of the at least one non-solid sample.

13. Laboratory apparatus according to claim 11 or 12, which further comprises a firmware, which stores and provides processing data for controlling the solidification of the at least one non-solid sample by means of the control device.

14. Laboratory apparatus according to any of the claims 11 to 13, which is a thermocycler, and wherein the work step comprises the repetitive thermal cycling of a sample solution, which comprises PCR-reagents.

15. Laboratory apparatus according to any of the claims 11 to 14, which further comprises
a sample block device for accommodating at least one sample vessel, which contains the at least one sample, and which forms the first temperature section,
at least one tempering device, which forms the at least one heat transport device,
a heat sink device, which forms the second temperature section,
wherein the at least one tempering device is arranged between the sample block device and the heat sink device.
